(19) United States
(12) Patent Application Publication
(10) Pub. No.: US 2024/0000852 A1
(43) Pub. Date: Jan. 4, 2024

(54) THERAPEUTIC USE OF CELL-FREE FAT EXTRACT FOR OSTEOPOROSIS

(71) Applicant: SHANGHAI SEME CELL TECHNOLOGY CO., LTD., Shanghai (CN)

(72) Inventors: Zhifeng YU, Shanghai (CN); Mingming XU, Shanghai (CN); Wenjie ZHANG, Shanghai (CN)

(21) Appl. No.: 18/039,639

(22) PCT Filed: Nov. 30, 2021
(86) PCT No.: PCT/CN2021/134637

§ 371 Date: May 31, 2023

(30) Foreign Application Priority Data
Dec. 1, 2020 (CN) 202011388384.0

(51) Int. Cl. A61K 35/35; A61P 19/10

(52) U.S. Cl. CPC A61K 35/35; A61P 19/10

(57) ABSTRACT

A cell-free fat extract is used for the preparation of a composition or a formulation. The composition or the formulation is effective in (i) preventing and/or treating osteoporosis; (ii) increasing bone volume fraction; (iii) increasing trabecular number; (iv) increasing trabecular connectivity density; (v) increasing bone mineral density; (vi) increasing trabecular thickness; and/or (vii) reducing trabecular separation.

### TECHNICAL FIELD

The invention relates to the field of medicine, in particular to the therapeutic use of cell-free fat extract for osteoporosis.

### BACKGROUND

Osteoporosis (OP) is a systemic bone disease due to decreased bone density and bone quality, and destruction of bone microstructure caused by a variety of reasons, resulting in increased bone fragility and thus susceptibility to fracture. It is one of the leading causes of fractures in individuals over 50 years of age and can cause serious and complex sequelae, both physically and psychologically, with risks such as increased relative mortality. Osteoporosis is divided into two categories: primary and secondary; primary osteoporosis is divided into three types: postmenopausal osteoporosis (type I), senile osteoporosis (type II) and idiopathic osteoporosis (including adolescent type). Postmenopausal osteoporosis generally occurs in women within 5 to 10 years after menopause; senile osteoporosis generally refers to osteoporosis that occurs after the age of 70; and idiopathic osteoporosis mainly occurs in adolescents, and the etiology is still unknown.

However, the therapeutic effect of the drugs in the prior art on osteoporosis is not ideal, and therefore, there is a need in the art to develop a drug that can effectively treat osteoporosis.

### SUMMARY OF THE INVENTION

The purpose of the present invention is to provide a use of a cell-free fat extract for preventing and/or treating osteoporosis.

A first aspect of the present invention provides a use of a cell-free fat extract in the preparation of composition or preparation for one or more uses selected from the group consisting of: (i) preventing and/or treating osteoporosis; (ii) increasing bone volume fraction; (iii) increasing bone trabecular number; (iv) increasing bone trabecular junction density; (v) increasing bone mineral density; (vi) increasing trabecular thickness; and/or (vii) reducing trabecular separation.

In another preferred embodiment, the osteoporosis includes disuse osteoporosis.

In another preferred embodiment, the prevention and/or treatment of osteoporosis is preventing and/or treating by one or more ways selected from the group consisting of:
(a) improving bone density;
(b) improving bone mass; and/or
(b) reducing bone loss.

In another preferred embodiment, the prevention and/or treatment of osteoporosis is preventing and/or treating by one or more ways selected from the group consisting of:
(ii) increasing bone volume fraction;
(iii) increasing bone trabecular number;
(iv) increasing bone trabecular junction density;
(v) increasing bone mineral density;
(vi) increasing trabecular thickness; and/or
(vii) reducing trabecular separation.

In another preferred embodiment, the bone is selected from the group consisting of tibia, femur, cervical vertebra, lumbar vertebra, thoracic vertebra, knee bone, hip bone, or a combination thereof.

In another preferred embodiment, the bone volume fraction is bone volume/total bone tissue volume.

In another preferred embodiment, the bone volume fraction comprises a bone volume fraction of cancellous bone.

In another preferred embodiment, the cell-free fat extract is a cell-free fat extract obtained from fat in human or non-human mammal.

In another preferred embodiment, the non-human mammal is a monkey, an orangutan, a cow, a pig, a dog, a sheep, a rat or a rabbit.

In another preferred embodiment, the composition or preparation comprises a pharmaceutical composition or preparation, a food composition or preparation, a nutraceutical composition or preparation, or a dietary supplement.

In another preferred embodiment, the composition or preparation further comprises a pharmaceutically, food, nutraceutical or a dietary acceptable carrier.

In another preferred embodiment, the dosage form of the composition or preparation is an oral preparation, a topical preparation or an injection preparation.

In another preferred embodiment, the injection preparation is intravenous injection or intramuscular injection.

In another preferred embodiment, the dosage form of the composition or preparation is a solid dosage form, a semi-solid dosage form, or a liquid dosage form, such as a solution, gel, cream, emulsion, ointment, cream, paste, cake, powder, patch, etc.

In another preferred embodiment, the dosage form of the composition or preparation is a powder, a granule, a capsule, an injection, a tincture, an oral liquid, a tablet or a lozenge.

In another preferred embodiment, the composition or preparation is administered by external, topical, or subcutaneous injection.

In another preferred embodiment, the cell-free fat extract is free of cell and free of lipid droplet.

In another preferred embodiment, the lipid droplets are oil droplets released after the fat cells are broken.

In another preferred embodiment, the “lipid droplet-free” means that the volume of oil droplets in the cell-free fat extract is less than 1%, preferably less than 0.5%, more preferably less than 0.1% in total liquid percentage.

In another preferred embodiment, the cells are selected from the group consisting of endothelial cells, adipose stem cells, macrophagocytic cells, stromal cells.

In another preferred embodiment, the “cell-free” means that the average number of cells in 1 ml of cell-free fat extract is ≤1, preferably ≤0.5, more preferably ≤0.1, or 0.

In another preferred embodiment, the cell-free fat extract is a naturally obtained nano-fat extract with additive-free.

In another preferred embodiment, the “additive-free” means that no solution, solvent, small molecule, chemical agent, and biological additive are added during the preparation of the fat extract except for the rinsing step.

In another preferred embodiment, the cell-free fat extract is obtained by centrifuging the fat tissue after emulsification.

In another preferred embodiment, the cell-free fat extract contains one or more components selected from the group consisting of: IGF-1, BDNF, GDNF, TGF-β1, HGF, bFGF, VEGF, TGF-β1, PDGF, EGF, NT-3, GH, G-CSF, and a combination thereof.

In another preferred embodiment, the cell-free fat extract contains, but is not limited to, one or more components selected from the group consisting of IGF-1, BDNF, GDNF, bFGF, VEGF, TGF-β1, HGF, PDGF, and a combination thereof.

In another preferred embodiment, the cell-free fat extract is a cell-free fat extract liquid.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

In another preferred embodiment, the cell-free fat extract is prepared by the following method:
(1) providing an fatty tissue raw material, crushing the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the primary fat extract, thereby obtaining a cell-free fat extract.

The second aspect of the present invention provides a method for preparing cell-free fat extract comprising the steps of:
(1) providing an fatty tissue raw material, crushing the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the primary fat extract, thereby obtaining a cell-free fat extract.

In another preferred embodiment, the cell-free fat extract is as described in the first aspect of the present invention.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, the temperature of the centrifugation is 2-6° C.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, the temperature of the centrifugation is 2-6° C.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the fat extract.

In another preferred embodiment, in the step (6), the filtration and sterilization are carried out through a filter (such as a 0.22 μm microporous filter membrane).

In another preferred embodiment, the filter is a microporous membrane filter.

In another preferred embodiment, the pore size of the microporous filter membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtration and de-bacterial is carried out by first filtering through a first filter that can filter cells, and then through a second filter (such as a 0.22 μm filter) that can filter pathogens (such as bacteria).

In another preferred embodiment, the step (6) further includes subpackaging the fat extract to form a subpackaging product. The subpacked extract can be stored at −20° C. for later use; it can be used directly after thawing at low temperature (e. g. −4° C.) or at normal temperature, or stored at low temperature (e. g. 4° C.) for a period of time for later use after thawing.

A third aspect of the present invention provides a cell-free fat extract, the cell-free fat extract is obtained by the method described in the second aspect of the present invention.

The fourth aspect of the present invention provides a composition or preparation, and the composition or preparation comprises (a) a cell-free fat extract as described in the third aspect of the present invention; and (b) a pharmaceutically, food, nutraceutical or dietary acceptable carrier or excipient.

In another preferred embodiment, the composition is a pharmaceutical composition, a food composition, a nutraceutical composition or a dietary supplement.

In another preferred embodiment, the dosage form of the composition or preparation is an oral preparation, a topical preparation or an injection preparation.

In another preferred embodiment, the dosage form of the composition or preparation is a powder, a granule, a capsule, an injection, a tincture, an oral liquid, a tablet or a lozenge.

In another preferred embodiment, the injection is an intravenous or intramuscular injection.

In another preferred embodiment, the dosage form of the composition or preparation is a solid dosage form, a semi-solid dosage form, or a liquid dosage form, such as a solution, gel, cream, emulsion, ointment, cream, paste, cake, powder, patch, etc.

In another preferred embodiment, in the composition or preparation, the mass percentage of the cell-free fat extract is 5 wt %, preferably 1-20 wt %, based on the total weight of the composition or preparation.

The fifth aspect of the present invention provides a method of preparing a composition or preparation according to the fourth aspect of the present invention comprising the step of: mixing the cell-free fat extract according to the third aspect of the present invention with a pharmaceutically, food, nutraceutical or dietary acceptable carrier or excipient to form the composition or preparation.

The sixth aspect of the present invention provides a method of (i) preventing and/or treating osteoporosis; (ii) increasing bone volume fraction; (iii) increasing bone trabecular number; (iv) increasing bone trabecular junction density; (v) increasing bone mineral density; (vi) increasing trabecular thickness; and/or (vii) reducing trabecular separation, comprising administering the cell-free fat extract according to the third aspect of the invention to a subject in need thereof.

In another preferred embodiment, the subject is a human or non-human mammal.

In another preferred embodiment, the non-human mammal comprises a rodent, such as a rat, a mouse.

It should be understood that within the scope of the present invention, the above-mentioned technical features of the present invention and the technical features specifically described in the following (such as examples) can be combined with each other to form a new or preferred technical solution. Limited to space, it will not be repeated here.

### DESCRIPTION OF THE DRAWINGS

FIG. 1 shows the effect of CEFFE on BMM cell viability.

FIG. 2 shows the effect of CEFFE on the osteoclast differentiation of BMM detected by TRAP staining.

FIG. 3 shows the effect of CEFFE on the expression of osteoclast differentiation-related mRNA in the osteoclast differentiation of BMM detected by QPCR.

FIG. 4 shows the effect of CEFFE on osteoclast differentiation-related mRNA expression in BMM osteoclast differentiation detected by QPCR.

FIG. 5 is micro CT analysis of cancellous bone and cortical bone, wherein TS refers to micro CT analysis of bone after osteoporosis modeling; CEFFE refers to CEFFE treatment for osteoporosis after modeling.

### DETAILED DESCRIPTION OF EMBODIMENTS

As a result of extensive and intensive studies, a cell-free fat extract with an excellent therapeutic effect on osteoporosis has been developed for the first time. On this basis, the present invention is completed.

Terms

Unless otherwise defined, all technical and scientific terms used herein have the same meaning as generally understood by those skilled in the art to which the present invention belongs.

As used herein, the terms “include”, “contain” and “comprise” are used interchangeably, including not only open definitions, but also semi-closed, and closed definitions. In other words, the term includes “consisting of” and “consisting essentially”.

As used herein, the terms “cell-free fat extract”, “cell free fat extract” and “CEFFE” are used interchangeably.

In the present invention, the term “prevention” means a method of preventing the onset of a disease and/or its accompanying symptoms or protecting a subject from developing the disease. The “prevention” used herein also includes delaying the onset of the disease and/or its accompanying symptoms and reducing the risk of disease in the subject.

The “treatment” described in the present invention includes delaying and terminating the progression of the disease, or eliminating the disease, and does not require 100% inhibition, elimination and reversal. In some embodiments, the composition or pharmaceutical composition of the present invention reduces, inhibits, and/or reverses osteoporosis, for example, by at least about 10%, by at least about 30%, by at least about 50%, or by at least about 80%, compared to the levels observed in the absence of the composition, kit, food or health kit, combination of active ingredients, described herein.

As used herein, the term “IGF-1” is called insulin-like growth factors-1.

As used herein, the term “BDNF” is called brain-derived neurotrophic factor (BDNF).

As used herein, the term “GDNF” is called glialcellline-derived neurotrophic factor.

As used herein, the term “bFGF” is called basic fibroblast growth factor.

As used herein, the term “VEGF” is called vascular endothelial growth factor.

As used herein, the term “TGF-01” is called transforming growth factor-β1.

As used herein, the term “HGF” is called hepatocyte growth factor.

As used herein, the term “PDGF” is called platelet derived growth factor.

As used herein, the term “EGF” is called Epidermal Growth Factor.

As used herein, the term “NT-3” is called neurotrophins-3.

As used herein, the term “GH” is called Growth Hormone.

As used herein, the term “G-CSF” is called granulocyte colony stimulating factor.

Cell Free Fat Extract (CEFFE) and Preparation Method Thereof

As used herein, the terms “cell-free fat extract of the present invention”, “extract of the present invention”, “fat extract of the present invention” and the like are used interchangeably to refer to an extract (or extract liquid) derived from fatty tissue prepared without adding any solutions, solvents, small molecules, chemicals, and biological additives during the preparation of the fat extract (other than the rinsing step). A typical process for preparing an extract of the present invention is as described above in the second aspect of the present invention. In addition, it should be understood that although the extract of the present invention does not need to add any additives (or additive ingredients) during the preparation process, some or a small amount of a safe substance (such as a small amount of water) that does not negatively or adversely affect the activity of the extract of the present invention can also be added.

The cell-free fat extract of the present invention can be derived from human fatty tissue, which is purified from nano-fat by removing oil and cell/extracellular matrix components after centrifugation, and is a liquid with cell-free, easy-to-prepare and rich in various growth factors.

In a preferred embodiment of the present invention, the cell-free fat extract is a cell-free fat extract liquid.

The cell-free fat extract described in the present invention may include a variety of cytokines. Typically, the cell-free fat extract comprises one or more of IGF-1, BDNF, GDNF, TGF-β1, HGF, bFGF, VEGF, TGF-β1, PDGF, EGF, NT-3, GH, and G-CSF.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

Preferably, the cell-free fat extract of the present invention is obtained by the method as described above in the second aspect of the present invention.

Typically, the cell-free fat extract described in the present invention is prepared by the following methods:
(1) providing an fatty tissue raw material, crushing the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the primary fat extract, thereby obtaining a cell-free fat extract.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the time of the centrifugation is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is a method of crushing by a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the fat extract.

In another preferred embodiment, in the step (6), the filtration and de-bacterial are carried out through a filter (such as a 0.22 μm microporous filter membrane).

In another preferred embodiment, the filter is a microporous membrane filter. In another preferred embodiment, the pore size of the microporous filter membrane is μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtration and de-bacterial is carried out by first filtering through a first filter that can filter cells, and then through a second filter (such as a 0.22 μm filter) that can filter pathogens (such as bacteria).

In another preferred embodiment, the step (6) further includes subpackaging the fat extract to form a subpackaging product. The subpacked extract can be stored at −20° C. for later use; it can be used directly after thawing at low temperature (e. g. −4° C.) or at normal temperature, or stored at low temperature (e. g. 4° C.) for a period of time for later use after thawing.

Osteoporosis

Osteoporosis (OP) is a systemic bone disease due to decreased bone density and bone quality, and destruction of bone microstructure caused by a variety of reasons, resulting in increased bone fragility and thus susceptibility to fracture. It is one of the leading causes of fractures in individuals over 50 years of age and can cause serious and complex sequelae, both physically and psychologically, with risks such as increased relative mortality.

In a preferred embodiment of the present invention, the osteoporosis is disuse osteoporosis. Disuse osteoporosis is a reduction in bone mass caused by limited or impaired limb movement, resulting in osteoporosis. Disuse osteoporosis is a kind of secondary osteoporosis that is closely associated with physical inactivity and is common in clinical practice. Disuse osteoporosis is most likely to occur when there is paralysis in bed, limb fixation and limited mobility, and weightlessness (e. g., astronauts). It can be secondary to primary osteoporosis and aggravate its condition.

Use

The present invention provides a use of a cell-free fat extract for the preparation of a composition or preparation for one or more uses selected from the group consisting of: (i) preventing and/or treating osteoporosis; (ii) increasing bone volume fraction; (iii) increasing trabecular bone number; (iv) increasing trabecular bone junction density; (v) increasing bone mineral density; (vi) increasing trabecular thickness; and/or (vii) decreasing trabecular separation.

In a preferred embodiment of the present invention, the osteoporosis includes disuse osteoporosis.

In another preferred embodiment of the present invention, preventing and/or treating osteoporosis is by one or more ways selected from the group consisting of:
(a) improving bone density;
(b) improving bone mass; and/or
(b) reducing bone loss.

In another preferred embodiment of the present invention, preventing and/or treating osteoporosis is by one or more ways selected from the group consisting of:
(ii) increasing bone volume fraction;
(iii) increasing bone trabecular number;
(iv) increasing bone trabecular junction density;
(v) increasing bone mineral density;
(vi) increasing trabecular thickness; and/or
(vii) reducing trabecular separation.

In another preferred embodiment of the present invention, the bone is selected from the group consisting of tibia, femur, cervical vertebra, lumbar vertebra, thoracic vertebra, knee bone, hip bone, and a combination thereof.

In another preferred embodiment, the bone volume fraction is bone volume/total bone tissue volume.

In another preferred embodiment, the bone volume fraction comprises a bone volume fraction of cancellous bone.

Composition and Administration

The compositions described herein include, but are not limited to, pharmaceutical compositions, food compositions, nutraceutical compositions, dietary supplements, and the like.

Typically, the cell-free fat extract of the present invention can be prepared as pharmaceutical compositions in dosage forms such as tablets, capsules, powders, microgranules, solutions, lozenges, jellies, creams, spiritus, suspensions, tinctures, mud dressings, liniment, lotions, and aerosols, etc. Pharmaceutical compositions can be prepared by commonly known preparation techniques, and suitable pharmaceutical additives can be added to the drug.

The compositions of the present invention can also include pharmaceutically, food, nutraceutical or dietary acceptable carriers. “Pharmaceutically, food, nutraceutical or dietary acceptable carriers” means one or more compatible solid or liquid filler or gel substances that are suitable for human use and must have sufficient purity and sufficiently low toxicity. “Compatibility” herein refers to the ability of components of a composition to blend with the compounds of the invention and with each without significantly reducing the efficacy of the compounds. Examples of pharmaceutically, food, nutraceutical or dietary acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, pyrogen-free water, etc.

The methods of administration of the compositions of the present invention are not particularly limited, and representative methods of administration include, but are not limited to, oral, parenteral (intravenous, intramuscular), topical administration, preferably oral administration and injection administration.

The dosage form of the composition or preparation described in the present invention is an oral preparation, a topical preparation or an injection preparation. Typically, solid dosage forms for oral administration include capsules, tablets, pills, powders and granules.

In these solid dosage forms, the active compounds is mixed with at least one conventional inert excipient (or carrier), such as sodium citrate or dicalcium phosphate, or mixed with: (a) fillers or compatibilizers, e.g., starch, lactose, sucrose, glucose, mannitol and silicic acid; (b) binders, e.g., hydroxymethylcellulose, alginate, gelatin, polyvinylpyrrolidone, sucrose, and gum arabic; (c) humectants, e.g., glycerol; (d) disintegrants, e.g., agar, calcium carbonate, potato starch or tapioca starch, alginic acid, certain complex silicates, and sodium carbonate; (e) dissolution-retarding agents, e.g., paraffin; (f) absorption accelerators, e.g., quaternary amine compounds; (g) wetting agents, e.g., cetearyl alcohol and glycerol monostearate; (h) sorbents, e.g., kaolin; and (i) lubricants, e.g., talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules, pills and granules may be prepared using coating and shell materials such as casing and other materials well known in the art. They can contain opaque agents.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and flavors.

In addition to the active ingredient, the suspension may comprise suspending agents, such as ethoxylated isooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compounds of the invention for topical administration include ointments, powder, patches, sprays and inhalants. The active ingredient is mixed under sterile conditions with a physiologically acceptable carrier and any preservatives buffers or propellants as may be required.

The cell-free fat extract of the present invention can be administered alone, or in combination with other drugs for preventing and/or treating fatty liver and/or its complications.

When the composition is administered, a safe and effective amount of the cell-free fat extract of the present invention is administrated to a human or non-human animal in need of treatment (e. g., rat, mouse, dog, cat, cow, chicken, duck, etc.) at a dose that is pharmaceutically, food, nutraceutical or dietary acceptable to the effective administration. As used herein, the term “safe and effective amount” refers to an amount that produces function or activity to humans and/or animals and is acceptable to humans and/or animals. Those ordinary skilled in the art will understand that the “safe and effective amount” described may vary depending on the form of the pharmaceutical composition, the route of administration, the excipient of the drug used, the severity of the disease, and the combination with other drugs. For example, for a person with a body weight of 60 kg, the daily dose is usually 0.1 to 1000 mg, preferably 1 to 600 mg, more preferably 2 to 300 mg. Of course, the specific dosage should also consider the route of administration, the patient's health and other factors, which are within the skill range of skilled doctors.

The main advantages of the present invention include:

1. The present invention is the first to discover that cell-free fat extracts have an excellent therapeutic effect on osteoporosis.

2. The cell-free fat extract of the present invention is a cell-free component that avoids cell-related problems in clinical applications, including, for example, genetic stability of cells after processing, cell viability and survival rate after injection, multiple dosing storage of cells, and immunogenicity of cells when using allogeneic fat, and the cell-free fat extract of the present invention has the advantages of higher safety and lower side effects in the preparation of drugs for preventing and treating osteoporosis.

The present invention is further described below in conjunction with specific examples. It is to be understood that these examples are intended to illustrate the invention only and not to limit the scope of the invention. The following examples do not indicate the specific conditions of the experimental method, usually according to the conventional conditions, or according to the conditions recommended by the manufacturer. Unless otherwise specified, percentages and parts are calculated by weight.

### Example 1

1. Methods

1.1. Preparation of Cell Free Fat Extract (CEFFE)

Fat is obtained by volunteers with informed consent. The preparation method of cell free fat tissue extract is as follows:

(1) Fatty tissue was obtained from 6 healthy women who underwent conventional liposuction, with an average age of 31 years (24-36 years). After anesthesia with local injection of swelling solution, a 3 mm liposuction aspiration cannula with a large lateral hole (2 mm×7 mm) connected to a 20 mL syringe was used, and the obtained fat was manually aspirated radially under negative pressure, and the fat was stood upright, and after removal of the swelling solution, it was rinsed 3 times with saline.

(2) The rinsed fatty tissue was taken, placed in a centrifuge tube, then placed in a centrifuge, and centrifuged at 1200 g 4° C. for 3 minutes to obtain a layered mixture.

(3) For the layered mixture, the upper oil layer and the lower water layer were removed and the intermediate layer (i.e. the fat layer containing fat cells) was collected.

(4) For the intermediate layer, two 10 ml syringes connected to a tee tube were pushed repeatedly and uniformly for 30 times, thus performing mechanical emulsification and obtaining a mechanically emulsified fat mixture (also called nano-fat).

(5) The mechanically emulsified fat mixture was placed into a −80° C. refrigerator for freezing, and then thawed in a 37° C. water bath, and after a single freeze-thaw cycle, the thawed fat mixture was centrifuged at 1200 g 4° C. for 5 minutes to obtain a layered mixture, which was divided into 4 layers, the first layer being the oil layer, the second layer being the residual fatty tissue layer, the third layer being the liquid layer, and the fourth layer being the cell/tissue debris precipitation layer, the oil layer and the residual fatty tissue layer were removed and the liquid layer was aspirated, avoiding contamination of the cell/tissue debris precipitation layer during the aspiration process, resulting in a primary fat extract.

(6) The obtained primary fat extract was filtered and de-bacterized through a 0.22 μm filter, thereby sterilizing and removing any live cells that may have been mixed, resulting in the cell-free fat extract (CEFFE) that was subpackaged and stored frozen at −20° C. and thawed at 4° C. when used.

For the cell-free fat extract prepared, the content of cytokines, including IGF-1, BDNF, GDNF, bFGF, VEGF, TGF-β1, HGF and PDGF, was detected by ELISA immunosorbent assay kit. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), and PDGF (179.9 pg/ml).

1.2 Effect of CEFFE on the Proliferation of BMM in Mice

Mouse-derived BMM (Bone Marrow-derived Macrophages, BMM) cells were used and inoculated in two 96-well plates. After 12 h, the cells were walled and the fluid were changed by adding F12 medium containing different concentrations of CEFFE (0-10 wt %). At 24 h and 48 h, the wells were changed to F12 medium containing 10% cck-8 reagent and placed in an incubator at 37° C. for 2 h. The absorbance at 450 nm was measured using an microplate reader (650 nm was chosen as a reference).

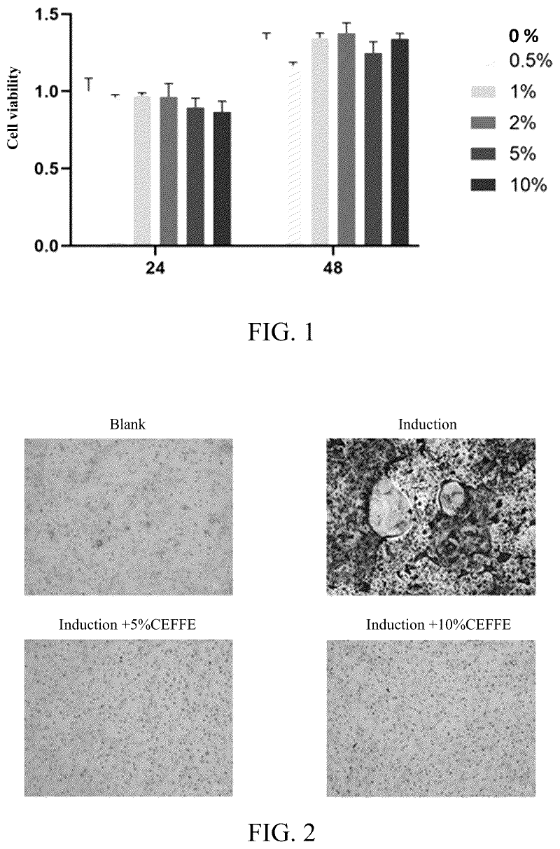

The results of CCK-8 detection for cell viability are shown in FIG. 1, which shows that CEFFE has no significant effect on BMM cell proliferation within a certain concentration range (0-10 wt %).

1.3 Effect of CEFFE on Osteoclast Differentiation of BMM in Mice

Mouse-derived BMM cells were grown in 12-well and 24-well plates, respectively. After 12 h, the cells were walled and the fluid were changed by adding osteoclast induction medium containing different concentrations of CEFFE (0-10 wt %), the medium were changed every two days with new CEFFE-containing medium, and after six days, RNA was extracted from each group of cells and TRAP staining was performed.

(1) The effect of CEFFE on osteoclast differentiation of BMM detected by TRAP (anti-tartrate phosphatase) staining is shown in FIG. 2, which shows that the Blank group had no osteoclast formation without the addition of RANKL to the culture medium; Control group, six days after induction with the addition of RANKL (Receptor Activator of Nuclear Factor-KB Ligand) in culture medium, multinucleated osteoclast cells was formed; however, two groups of the Induction +5% CEFFE/10% CEFFE group did not induce BMM osteoclast formation after adding RANKL and different concentrations of CEFFE at the same time, and the Induction refers to the induction by adding RANKL.

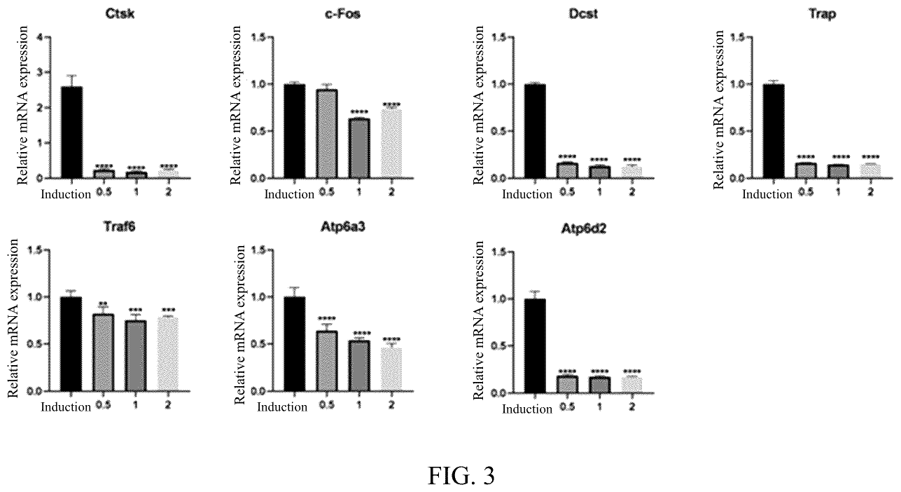

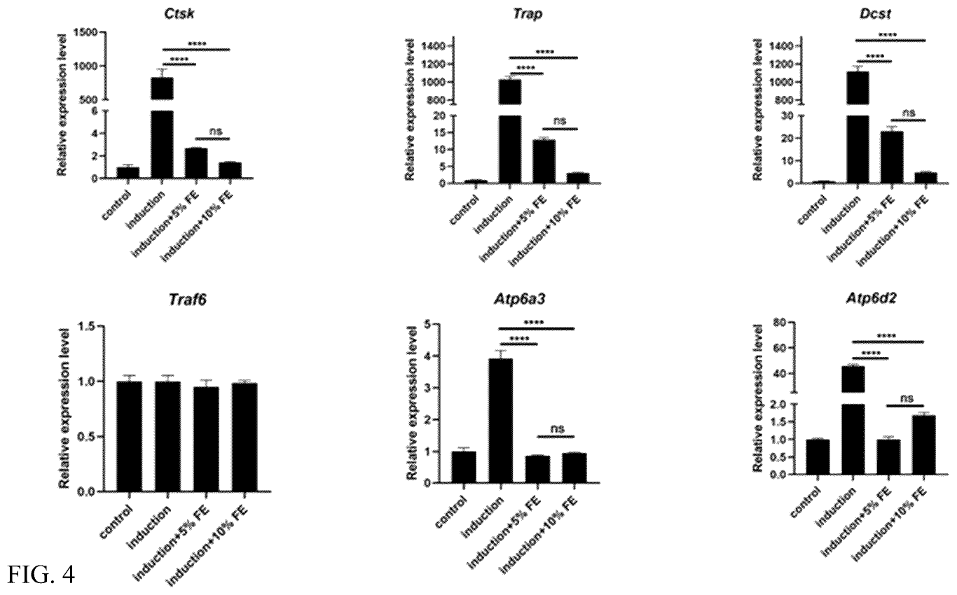

(2) The effect of different levels of CEFFE (0.5% and 1% and 2%) on the expression of osteoclast differentiation-related mRNA in BMM was detected by QPCR (quantitative real-time fluorescence PCR) assay as shown in FIGS. 3 and 4. Whether under the conditions of adding CEFFE alone (FIG. 3) or osteoclast induction with the addition of CEFFE (FIG. 4), the expression of osteoclast differentiation-related genes Ctsk, c-Fos, Dcst, Trap, Traf6, Atp6a3 and Atp6d2 were significantly reduced, and CEFFE significantly inhibited osteoclast differentiation of mouse BMM cells at 0.5 wt % compared to the RANKL-induced control group.

1.4 Establishment of Osteoporosis Model in Mice

After 8-week-old C57 male mice were anaesthetized with pentobarbital, the tails of the mice were wiped clean with alcohol cotton balls, and the tails of the mice were attached to one end of the string using 415 strong adhesive (Abeta Instant Adhesive), and then the other end of the string was fixed to the top of the cage so that the angle of the mice's body to the ground was greater than 30° to establish the disuse osteoporosis mouse model. After three weeks of tail hanging, the bone loss in the hind limbs was obvious, so that the osteoporosis model was successfully established.

In vivo, the mouse tail-hanged model caused the mouse hind limbs in weightlessness environment, resulting in bone loss of hind limbs. The effect of CEFFE on bone loss in the hind limbs of tail-suspended mice was evaluated by intraperitoneal injection of CEFFE (every 3 days/time, a total of 7 times, 250 μl for each time) after successful moulding. The effect of CEFFE on bone loss in the hind limbs of tail-hanged mice was evaluated after the samples were taken after 4 weeks.

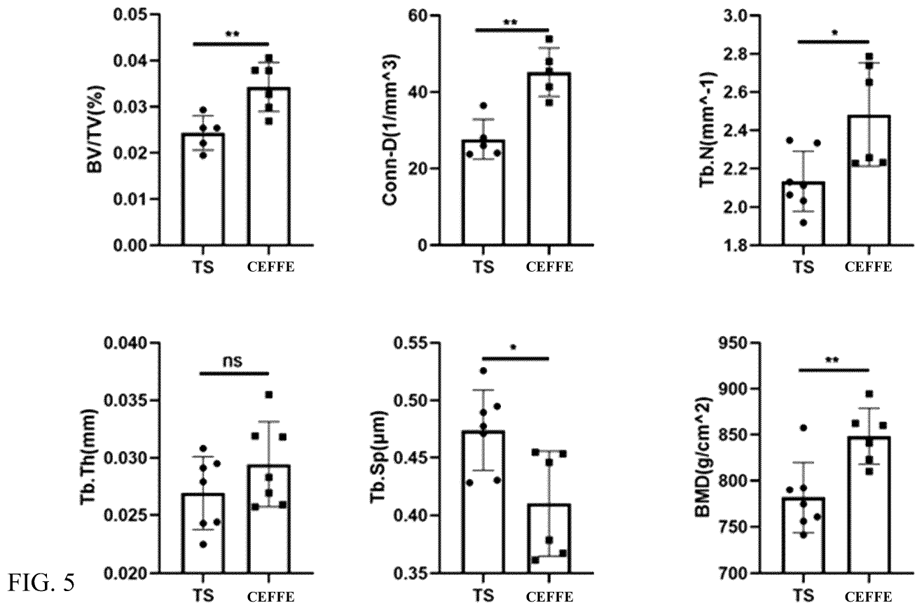

The results of micro CT analysis of cancellous and cortical bone of the tibia were shown in FIG. 5. The CEFFE can improve the structure of hind limb bone, reduce bone loss and diminish bone resorption: in the CEFFE-treated group, BV/TV (bone volume/total bone tissue volume, i.e. bone volume fraction) of cancellous bone increased, Tb.N (bone trabecular number) increased, Conn-Dens (Conn-D, junction density) increased, Tb.sp (bone trabecular separation) decreased, Tb.Th (bone trabecular average thickness) increased, and BMD (bone mineral density) increased, resulting in an excellent therapeutic effect on osteoporosis.

All documents referred to in the present invention are incorporated by reference herein as if each document is individually incorporated by reference. Further, it should be understood that upon reading the above teaching of the present invention, various variations or modifications may be made to the present invention by those skilled in the art, and those equivalents also fall within the scope defined by the appended claims of the present application.

1. A method for: (i) preventing and/or treating osteoporosis; (ii) increasing bone volume fraction; (iii) increasing trabecular bone number; (iv) increasing trabecular bone junction density; (v) increasing bone mineral density; (vi) increasing trabecular thickness; and/or (vii) decreasing trabecular separation, comprising the step of administering a cell-free fat extract or a composition or preparation containing the cell-free fat extract to a subject in need thereof.

2. The method according to claim 1, wherein the prevention and/or treatment of osteoporosis is preventing and/or treating by one or more ways selected from the group consisting of:
(a) improving bone density;
(b) improving bone mass; and/or
(b) reducing bone loss.

3. The method according to claim 1, wherein the prevention and/or treatment of osteoporosis is preventing and/or treating by one or more ways selected from the group consisting of:
(ii) increasing bone volume fraction;
(iii) increasing bone trabecular number;
(iv) increasing bone trabecular junction density;
(v) increasing bone mineral density;
(vi) increasing trabecular thickness; and/or
(vii) reducing trabecular separation.

4. The method according to claim 1, wherein the bone is selected from the group consisting of tibia, femur, cervical vertebra, lumbar vertebra, thoracic vertebra, knee bone, hip bone, and a combination thereof.

5. The method according to claim 1, wherein the osteoporosis comprises disuse osteoporosis.

6. The method according to claim 1, wherein the cell-free fat extract comprises one or more components selected from the group consisting of IGF-1, BDNF, GDNF, TGF-β1, HGF, bFGF, VEGF, TGF-β1, HGF, PDGF, EGF, NT-3, GH, G-CSF, and a combination thereof.

7. The method according to claim 6, wherein the cell-free fat extract comprises one or more features selected from the group consisting of
in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml;
in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml;
in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml;
in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml;
in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml;
in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml;
in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml; and/or
in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

8. The method according to claim 6, wherein the cell-free fat extract comprises one or more features selected from the group consisting of
the weight ratio of the IGF-1 to VEGF is 20-100:1, preferably 30-70:1, more preferably 40-60:1, and most preferably 45-55:1;
the weight ratio of BDNF to VEGF is 2-20:1, preferably 4-15:1, more preferably 6-12:1, and most preferably 8-9.5:1;
the weight ratio of GDNF to VEGF is 2-20:1, preferably 4-15:1, more preferably 6-12:1, and most preferably 8.5-9.5:1;
the weight ratio of bFGF to VEGF is 0.2-8:1, preferably 0.5-5:1, more preferably 0.6-2:1, more preferably 0.8-1.6:1, and most preferably 1-1.5:1;
the weight ratio of TGF-β1 to VEGF is 1-20:1, preferably 1-15:1, more preferably 1-10:1, more preferably 2-8:1, more preferably 4-6:1;
the weight ratio of HGF to VEGF is 1-20:1, preferably 1-15:1, more preferably 1-10:1, more preferably 2-8:1, more preferably 4-5.5:1; and/or
the weight ratio of PDGF to VEGF is 0.1-3:1, preferably 0.2-2:1, more preferably 0.4-1.5:1, and most preferably 0.7-1.2:1.

9. The method according to claim 1, wherein the cell-free fat extract is prepared by the following method:
(1) providing an fatty tissue raw material, crushing the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano-fat);
(5) centrifuging the emulsified fat mixture, thereby obtaining an intermediate liquid layer, i.e. a primary fat extract; and
(6) filtering and de-bacterizing the fat primary extract, thereby obtaining a cell-free fat extract.

10. The method according to claim 1, wherein the composition or preparation further comprises a pharmaceutically, food, nutraceutical or dietary acceptable carrier.

11. The method according to claim 1, wherein the composition or preparation is administrated by external, topical, or subcutaneous injection.

12. (canceled)
